(19) United States
(12) Patent Application Publication
(10) Pub. No.: US 2014/0246030 A1
(43) Pub. Date: Sep. 4, 2014

(54) ENDOSCOPIC BITE BLOCK SHIELD

(71) Applicants: AMITAB PURI, TUCSON, AZ (US); SARAH PURI, TUCSON, AZ (US)

(72) Inventors: AMITAB PURI, TUCSON, AZ (US); SARAH PURI, TUCSON, AZ (US)

(21) Appl. No.: 13/782,801

(22) Filed: Mar. 1, 2013

(51) Int. Cl. A61F 5/37

(52) U.S. Cl. CPC A61F 5/37
USPC 128/887

(57) ABSTRACT

A device for use during a procedure for inserting a medical device into an esophagus of a patient has a first shield attached to a bite block to protect health care providers from fluids emanating from the patient during the procedure.

### BACKGROUND

Embodiments of this disclosure relate generally to endoscopic bite blocks, and more particularly, to a shield for use with an endoscopic bite block to protect health care providers from secretions, arilization of secretions from coughing or the like, or other secretions/excretions from a mouth of a patient.

An endoscopic bite block generally is a medical device having a body section that may be positioned into the mouth between the teeth or dental ridges of a patient. The body section generally has a central passageway which may be used to insert an endoscope, broncoscope or other type of insertion tube into the month and down the esophagus of the patient. The central channel is generally formed of a hard plastic which may be used to prevent the patient from biting down on the endoscope. A flared member may extend from both sides of the central passageway. The flared member may be used to prevent the body section from falling into the mouth of the patient.

Generally at the time of insertion, the patient may experience vomiting and or coughing sensation (hereinafter sensation). The sensation exacerbates as the insertion tube goes down the esophagus. This may cause the patient to cough and or expel secretions or saliva towards the health care provider.

Further, the patient's mouth may generate large amount of saliva due to the vomiting sensation. Accumulation of saliva in the mouth becomes bothersome for the patient and the procedure may have to come to halt so the patient cannot get rid of the excess saliva in his/her mouth. Prior art endoscopic bite blocks do not provide easy access for a suction device to removing the excess saliva during the endoscopy procedure.

As such, a need therefore exists to provide a device and method to overcome the above issues. The device would be used to protect health care providers from secretions from a mouth of a patient during an endoscopic, bronchoscopy, or like procedure. The device would further provide easy access for a suction or like device to be used during the endoscopic or like procedure.

### SUMMARY

A device for use during a procedure for inserting a medical device into an esophagus of a patient has a first shield attached to a bite block to protect health care providers from fluids emanating from the patient during the procedure.

A device for use during a procedure for inserting a medical device into an esophagus of a patient has a bite block. The bite block comprises: a front flange; a channel coupled to a central area of the front flange; and at least one auxiliary channel coupled to the channel. A first shield is attached to a bite block to protect health care providers from fluids emanating from the patient. The first shield comprises: a conical hollow body member coupled to the bite block; and a cover member formed over a front opening in the conical hollow body member, the cover member being perforated to allow the medical device to enter the conical hollow body member.

A device for use during a procedure for inserting a medical device into an esophagus of a patient has a bite block comprising: a front flange; a channel coupled to a central area of the front flange; and at least one auxiliary channel coupled to the channel. A first shield is attached to a bite block to protect health care providers from fluids emanating from the patient. A second shield is attached to the bite block to protect health care providers from fluids emanating from the patient.

### BRIEF DESCRIPTION OF THE DRAWINGS

Embodiments of the disclosure will become more fully understood from the detailed description and the accompanying drawings, wherein:

FIG. 1 is a perspective view of a bite block of the present invention with a protective shield removed;

FIG. 2 is a perspective view of the bite block of FIG. 1, with one embodiment of the protective shield;

FIG. 3 is a side view of the bite block of FIG. 2;

FIG. 4 is a rear view of the bite block of FIG. 2;

FIG. 5 is a perspective view of the bite block of FIG. 2 showing an endoscopic insertion tube being inserted therein, and an auxiliary device being inserted into an auxiliary channel;

FIG. 6 is a perspective view of the bite block of FIG. 2, further comprising a second protective shield; and

FIG. 7 is a perspective view of the bite block of FIG. 6, while placed in the patient's mouth.

### DETAILED DESCRIPTION

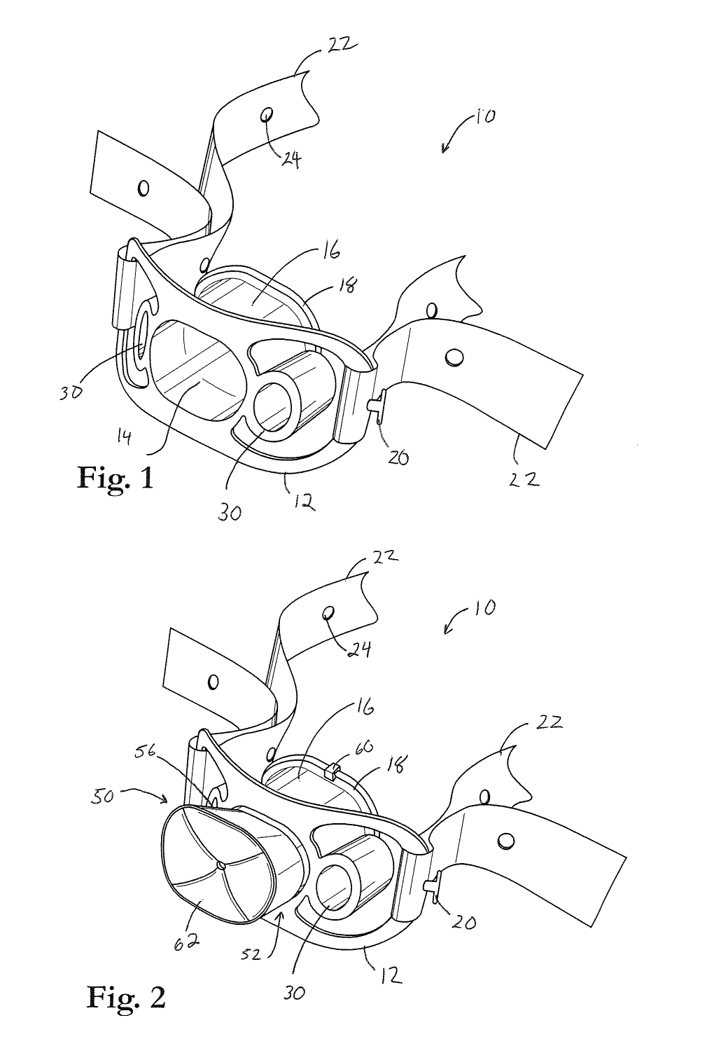

Referring to FIG. 1, a perspective view of a bite block 10 is shown. The bite block 10 may be used during medical procedures when endoscope, broncoscope or other type devices may be inserted into the month and down the esophagus of the patient.

The bite block 10 is shown as having a front flange 12. The front flange 12 may be used to cover a patient's facial structure to secure the bite block 10 and to prevent the bite block from falling into a mouth of a patient. In the present embodiment, the front flange 12 has a curved shape corresponding to fit the generally convex counters of the face and lips of the patient.

A channel 16 is coupled to a central area of the front flange 12. In accordance with one embodiment, the channel 16 and the front flange are integrally coupled. The channel 16 may be of different shapes. In the present embodiment, the channel 16 is oval in shape. However, this is shown as an example and should not be seen in a limiting scope. A rear flange 18 may be formed on a distal end of the channel 16. In accordance with one embodiment, the rear flange 18 may be positioned behind the patient's teeth.

On the front flange 12 and adjacent to the opening 16 may be at least one auxiliary channel 30. The auxiliary channels 30 may be integrally coupled to the front flange 12. Each of the auxiliary channels 30 extend rearward from the front flange 12. The auxiliary channels 30 may be the same length as the channel 16. However, in this embodiment of the bite block 10, the auxiliary channels 30 are shorter than the channel 16. In other embodiments of the bite block 10, the auxiliary channels 30 may be longer than the central channel 16.

The auxiliary channels 30 may have an oval cross section. However, the auxiliary channels 30 may be round, rectangular, or other suitable cross sections. Furthermore, the auxiliary channels 30 may be smaller compare to the opening 14. The auxiliary channels 30 may be configured to allow an instrument to be inserted therein. In the present embodiment, the auxiliary channels 30 may be used to insert a suction device in the patient's mouth during the endoscopy procedure. The suction device may be used to remove excess saliva from the patient's mouth.

In present embodiment of the bite block 10, at each side of the front flange 12, fastening apparatuses 20 may be placed. The fastening apparatuses 20 may be used to hold a tie member 22 to the bite block 10 to secure the bite block 10 on a patient's head. Although, the shape of these fastening apparatuses 20 is not of significance, here the fastening apparatuses 20 are T shaped anchors. A tie member 22 interfaces with the fastening apparatuses 20. The tie member 22 may have a plurality of holes 24 formed there through. The holes 24 may be used to secure the tie member 22 on the fastening apparatuses 20 and to adjust the length of the tie member 22.

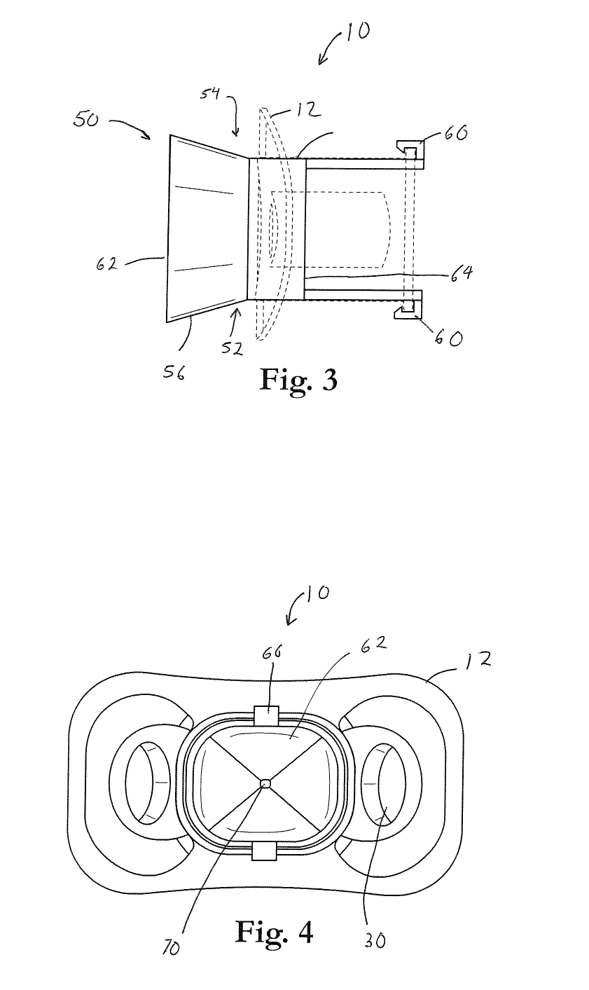

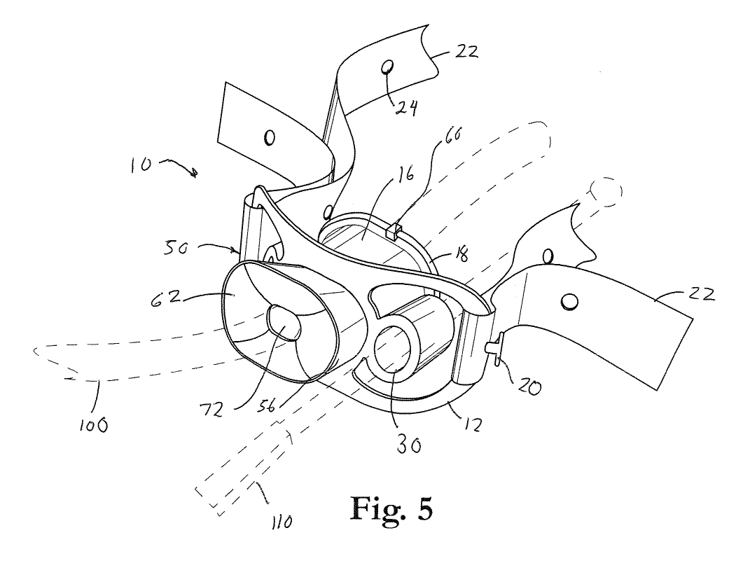

Referring now to FIGS. 2, 3, 4 and 5, a particular embodiment of the bite block 10 is shown further comprising a first shield 50. The first shield 50 may be used to protect health care providers from secretions, the arilization of secretions from coughing or the like, or other secretions/excretions (hereinafter secretions) that may exit from a mouth of a patient.

The first shield 50 is shown as having a hollow body 52. The hollow body 52 is inserted into the central channel 16. The hollow body 52 may be comprised of a plurality of segments 54. The plurality of segments 54 may comprise a tapered segment 56. Here, the tapered segment 56 may be used to safely direct the endoscopy tube into the patient's mouth. Adjacent the tapered segment 56, an extension segment 58 may be couple to the tapered segment 56 and extend therefrom. Here, the extension segment 58 integrally joins the tapered segment 56 and extended therefrom. The tapered segment 56 and the extension segment 58 may form diverse shapes. Here, the tapered segment 56 has a funnel shape while the extension segment 58 is a cylinder with an oval cross section. Further, the tapered segment 56 may transition into the extension segment 58, rather than breaking into the recess segment. In addition, at least one fastener 60 may integrally join to the extension segment 58 and extend therefrom. The fastening mechanisms 60 may be used to secure the first shield 50 to the bite block 10.

The hollow body 52 of the first shield 50 may have a cover 62 at one end, and an outlet end 64 at an opposing end. The cover 62 may be formed of a crossed break entry which would be punched through as an endoscopic tube enters the hollow body 52. The advantage of having the cover 62 with a breakable entry may be realized during the endoscopy procedure, because the cover 62 prevents salvia from the patient's mouth from exiting through hollow body 52 and soiling the health care provider.

The first shield 50 may be a separate device which may be secured to a pre-existing bite block or the bite block 10. To secure the first shield 50 to the bite block 10, the first shield 50 is inserted into the channel 16 of the bite block 10. The opening 14 may be sized to receive the extension segment 58 and at least one fastener 60 therein. Once the first shield 50 is received by the channel 16, the at least one fastener 60 of the first shield 50 may engage the rear flange 18. Here, in order to insert the tube receptacle 50 into the central channel 16, a user may be required to insert the first shield 50, from the outlet end 64 up to near the tapered segment into the opening 14 until the at least one fastening mechanism 60 snaps to the rear flange 18.

Once the first shield 50 is secured to the bite block 10, the bite block 10 and first shield 50 may be placed in the patient's mouth. The endoscopic insertion tube 100 may then be introduced into the first shield 50. An opening 70 in the center of the cover 62 of the tube receptacle may open up to a passage way 72 for the endoscopic instrument. Here the passage way 72 encloses around the endoscopy insertion tube 100, and hence prevents saliva accumulated in the patient's mouth (not shown) to exit out through the first shield 50. Adjacent to the channel 16, the auxiliary channels 30 may be utilized for insertion of at least one auxiliary instrument 110 in the patient's mouth. For example, the at least one auxiliary instrument 110 may be a suction device to remove excess saliva from the patient's mouth.

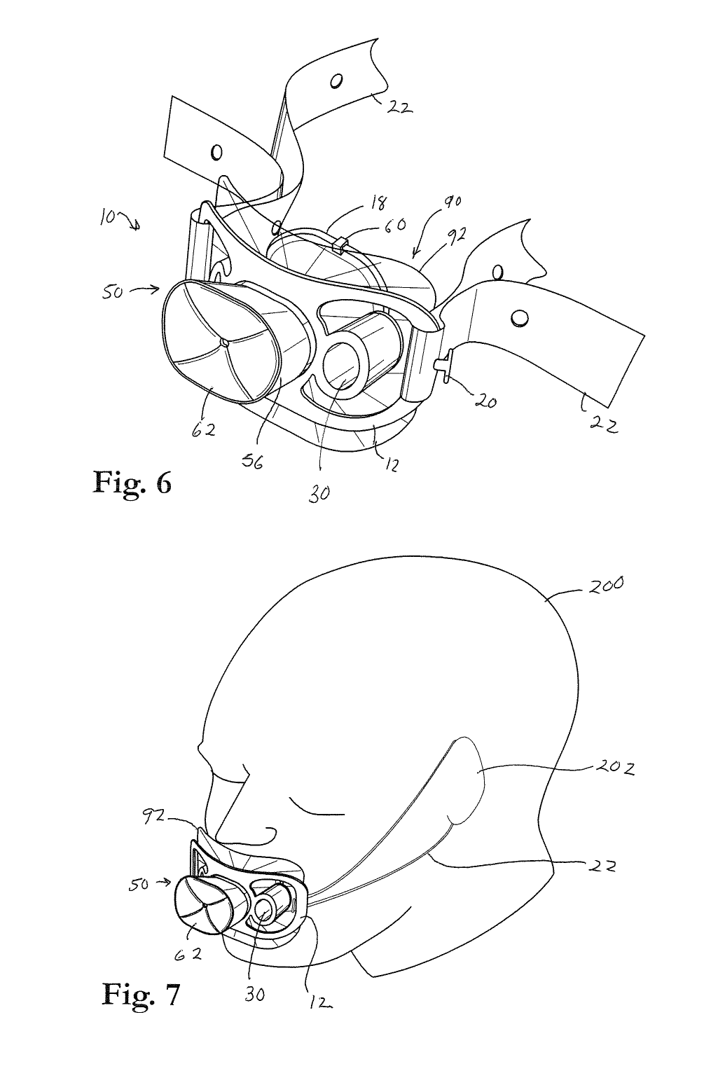

Referring now to FIGS. 6 and 7, a particular embodiment of the bite block 10 is shown further comprising a second shield 90 along with the first shield 50. While FIGS. 6 and 7 show the second shield 90 in combination with the first shield 50, the second shield 90 may be used alone with the bite block 10.

The second shield 90 may be interposed between the front flange 12 and the patient's mouth. The second shield 90 may be coupled to the front flange 12 or the central channel 16 or both. In the present embodiment, the second shield 90 is coupled to the central channel 16. In accordance with another embodiment, the second shield 90 maybe attached to an outer perimeter of the front flange 12.

The second shield 90 may be comprised of elastic and soft material similar to the material used in a surgical mask. The second shield 90 may be used to protect the health care provide from secretions from the patient during the endoscopy procedure. The second shield 90 may have a curved body 92 corresponding to the contour surface of the patient's face.

The second shield 90 may further include a plurality of passages corresponding to the channel 16 and the auxiliary channels 30 of the bite block 10. Here, the plurality of passages comprises three passages corresponding to the channel and the auxiliary channels 30. In some embodiments of the bite block 10, the second shield 90 may be an integral part of the bite block 10, while in other embodiments the protective shield 90 may be releasably coupled to the bite block 10.

The second shield 90 may have one or more straps 22. One strap 22 may be attached to opposing sides of the second shield 90. The straps 22 may be used to secure the second shield 90 to the head of the patient.

Referring now to FIG. 7, operation of the bite block 10 having the first shield 50 and the second shield 90 will be disclosed. In this embodiment of the bite block 10, there would be at least three openings toward the patient's mouth. One opening is provided through the channel 16 and two openings through the auxiliary channels 30. As shown here, the tie member 22 may engage the patient head 200, and hence maintain the bite block 10 in the patient's mouth. Here, the tie 22 member engages the patient's ears 202, thus pulling the bite block 50 toward the patient's mouth. The patient's teeth would be placed on the channel 16, and the rear flange 18 would be placed behind the patient's teeth. Here, the channel 16 in combination with the at least one auxiliary channel 30 may suppress the patient's tongue during the endoscopy procedure. In addition, the auxiliary channels 30 may be utilized for auxiliary medical devices to be inserted into the patient's mouth.

While embodiments of the disclosure have been described in terms of various specific embodiments, those skilled in the art will recognize that the embodiments of the disclosure may be practiced with modifications within the spirit and scope of the claims.

What is claimed is:

1. A device for use during a procedure for inserting a medical device into an esophagus of a patient comprising:
a first shield attached to a bite block to protect health care providers from fluids emanating from the patient.

2. The device of claim 1, wherein the bite block comprises:
a front flange;
a channel coupled to a central area of the front flange; and
at least one auxiliary channel coupled to the channel.

3. The device of claim 2, wherein the bite block further comprises:
at least two fastening apparatuses on a peripheral of the front flange; and
a tie member coupled with the at least two fastening apparatuses

4. The device of claim 1, wherein the first shield comprises:
a conical hollow body member coupled to the bite block; and
a cover member formed over a front opening in the conical hollow body member, the cover member being perforated to allow the medical device to enter the conical hollow body member.

5. The device of claim 5, further comprising at least one attachment member coupled to the conical hollow body member to removably couple the first shield to the bite block.

6. The device of claim 2, wherein the first shield comprises:
a conical hollow body member coupled to the channel of the bite block;
a cover member formed over a front opening in the conical hollow body member, the cover member being perforated to allow the medical device to enter the conical hollow body member; and
at least one attachment member coupled to the conical hollow body member to removably couple the first shield to the channel of the bite block.

7. The device of claim 1, further comprising a second shield coupled to the bite block.

8. The device of claim 6, wherein the second shield comprises a curved body member corresponding to a contour surface of a face of the patient.

9. The device of claim 7, wherein the second shield further comprises at least one strap coupled to an edge of the curved body member.

10. The device of claim 2, further comprising a second shield coupled to the bite block.

11. The device of claim 9, wherein the second shield comprises a curved body member corresponding to a contour surface of a face of the patient.

12. The device of claim 10, wherein the second shield further comprises at least one strap coupled to an edge of the curved body member.

13. A device for use during a procedure for inserting a medical device into an esophagus of a patient comprising:
a bite block comprising:
a front flange;
a channel coupled to a central area of the front flange; and
at least one auxiliary channel coupled to the channel; and
a first shield attached to a bite block to protect health care providers from fluids emanating from the patient, wherein the first shield comprises:
a conical hollow body member coupled to the bite block; and
a cover member formed over a front opening in the conical hollow body member, the cover member being perforated to allow the medical device to enter the conical hollow body member.

14. The device of claim 13, wherein the bite block further comprises:
at least two fastening apparatuses on a peripheral of the front flange; and
a tie member coupled with the at least two fastening apparatuses

15. The device of claim 13, further comprising a second shield coupled to the bite block.

16. The device of claim 15, wherein the second shield comprises a curved body member corresponding to a contour surface of a face of the patient.

17. The device of claim 16, wherein the second shield further comprises at least one strap coupled to an edge of the curved body member.

18. A device for use during a procedure for inserting a medical device into an esophagus of a patient comprising:
a bite block comprising:
a front flange;
a channel coupled to a central area of the front flange; and
at least one auxiliary channel coupled to the channel;
a first shield attached to the bite block to protect health care providers from fluids emanating from the patient; and
a second shield coupled to the bite block to protect health care providers from fluids emanating from the patient.

19. The device of claim 18, wherein the first shield comprises:
a conical hollow body member coupled to the bite block;
a cover member formed over a front opening in the conical hollow body member, the cover member being perforated to allow the medical device to enter the conical hollow body member; and
at least one attachment member coupled to the conical hollow body member to removably couple the first shield to the channel of the bite block.

20. The device of claim 18, wherein the second shield comprises a curved body member corresponding to a contour surface of a face of the patient.
